(19) United States
(12) Patent Application Publication
(10) Pub. No.: US 2026/0227304 A1
(43) Pub. Date: Aug. 6, 2026

(54) METHOD AND APPARATUS FOR THE CHARACTERIZATION OF A NON-HOMOGENEOUS SOLID GRANULAR MEDIUM

(71) Applicant: UNIVERSITA' DEGLI STUDI DI MILANO-BICOCCA, Milano (IT)

(72) Inventors: Valter MAGGI, Zelo Buon Persico (Lodi) (IT); Claudio ARTONI, Milano (IT); Marco Alberto Carlo POTENZA, Milano (IT)

(21) Appl. No.: 19/154,494

(22) PCT Filed: Feb. 8, 2024
(86) PCT No.: PCT/IB2024/051164

§ 371 Date: Aug. 7, 2025

(30) Foreign Application Priority Data
Feb. 9, 2023 (IT) 102023000002175

(51) Int. Cl. G01N 15/0205

(52) U.S. Cl. CPC G01N 15/0211

(57) ABSTRACT

A method for the optical characterization of a non-homogeneous solid granular medium, with an apparatus having a probe rod inserted into the non-homogeneous solid granular medium and a processor, the probe rod carrying at least one photoemitter and at least one photodetector, involves, by the photoemitter, emitting a probe light signal producing, upon interaction with the non-homogeneous solid granular medium, a return light signal scattered by the non-homogeneous solid granular medium and having intensity fluctuations deriving from local inhomogeneities or anisotropies of the non-homogeneous solid granular medium, and, by the photodetector, measuring a plurality of intensity values of the return light signal in a plurality of respective detection points, so as to detect the intensity fluctuations. The method further involves, by the processor, determining physical properties of the non-homogeneous solid granular medium by statistical analysis of the plurality of measured intensity values of the return light signal.

The present invention relates generally to techniques for characterizing the properties of a non-homogeneous solid medium, such as snow contained in a snowpack.

### BACKGROUND OF THE INVENTION

Snowpack is a mixture of ice crystals, liquid water, water vapor, air, and various other compounds and particles. The structure, composition, and chemical and physical characteristics are constantly changing due to multiple factors. The pressures induced by the accumulation of new snow cause a decrease in air content and a settling process of sintering by compaction. Other factors may include geothermal heat flux, thermal energy transmission in the snowpack, incident radiation, reflected radiation, fog, wind, nighttime irradiation under clear skies, and temperature.

There are three thermal gradient types in snow to which correspond three types of metamorphism: low gradient, which causes destructive metamorphism with decrease in crystal volume, medium gradient and high gradient, which cause constructive metamorphism with strong crystal growth. A weak gradient causes sintering of grains (on a time scales of days), while a medium gradient causes faceted particles to form, and finally a high gradient produces depth hoar grains.

The stratigraphic analysis of the snowpack is carried out by dividing the profile into basically horizontal layers according to the relative hardness. This is determined qualitatively through the hand test or quantitatively through electronic penetrometric probes (e.g. SnowMicroPen®).

For each layer, the shape, size, and water content of the snow crystals are also classified. The shape of the crystals is determined by following the guidelines of the International Classification for Seasonal Snow on the Ground, through a crystallographic tablet and lens.

Through stratigraphic analysis, it is possible to determine the weak layers along which an avalanche could be triggered and the more consolidated layers that bind the snowpack better. The crystals, having different shapes, may give rise to avalanches of different types.

The following is a list of grain types and the relevance thereof:
Partially decomposed precipitation particles form in the snowpack (usually near the surface) in dry snow. The crystals undergo a decrease in surface area that increases with the increase in temperature. Snow at this stage has a cohesion recovery by sintering after an initial drop in strength due to the decomposition process. These grains give rise to weakly cohesive avalanches or soft slab avalanches (especially if the particles are broken up by the wind).
Rounded grains are formed by the destructive metamorphism of the snow crystals. The strength is provided by the sintering of snow grains and increases with time. These grains are mainly responsible for slab avalanches (in the presence of weak layers under said slabs).
Faceted grains are formed by constructive metamorphism, and the strength decreases with the growth of the grains. These grains are responsible for the weak layers where slab avalanches may break off.
Depth hoar is formed at the base of the snowpack where the crystals undergo constructive metamorphism that leads them to have very large sizes and hollow cup shapes, striated prisms or chains. These grains are responsible for slab avalanches even at the base.
Melted forms form the characteristic clustered crystals (“corn”) and are associated with the percolation of water in the mantle or a process of melting and recrystallization (melt-freeze crusts). These grains form avalanches of wet or damp snow.
Ice layers are formed by the percolation of melted water in the snowpack (e.g., rain or meltwater) that refreezes. The process generally occurs most easily at the interfaces with layers of fine grains on top of coarse grains. The ice layers are strong, but the strength drops once the snow is completely wet.

Within the snowpack, the cohesion of the grains is the ability of snow grains to stick together by virtue of the intermolecular forces of attraction or bonds (necks) that have been created. Cohesion may then occur by felting, sintering, melting and refreezing, weak intermolecular bonds, and capillarity.

Snow, although flat and seemingly stationary, is always moving. Movements are divided into (1) slow: settling, sliding, creeping, slipping; (2) fast: avalanches. An avalanche is defined as a mass of snow that is set in motion instantaneously as a result of the breakage of the equilibrium conditions of the snowpack present on a sufficiently inclined slope due to natural or accidental causes and that precipitates downstream due to gravity under conditions of low friction. A decrease in strength and friction may be promoted by an increase in temperature or by the presence within the snowpack of weak or critical layers (crusts, hoar, faceted grains).

In nivology, stability is synonymous with balance. A snowpack is unstable when it is no longer able to maintain its mechanical equilibrium and/or change it according to its own transformations or imposed external conditions (overloading). The snowpack structure provides objective clues and signals of structural instability that may be used for predicting avalanches.

WO 2017/058929 A1 describes a sensor system for measuring the profile of a snowpack. This system is based on measuring the intensity value of scattered light collected by a photodetector placed on a probe rod. Such a measurement allows only the surface of the layer to be highlighted, but does not allow further information to be obtained.

Arnaud L. et al: “Measurement of vertical profiles of snow specific surface area with a 1 cm resolution using infrared reflectance: instrument description and validation”, Journal of glaciology, vol. 57, no. 201, 2011, pp. 17-29 and U.S. Pat. No. 6,957,593 B1 describe additional systems and methods for measuring the profile of a snowpack.

An object of the invention is to provide a method for performing a fuller characterization of the snowpack. More generally, another object of the invention is to make available a method that allows for the optical characterization of the properties of a non-homogeneous solid medium.

### SUMMARY OF THE INVENTION

For the aforementioned objects, a method for the optical characterization of a non-homogeneous solid granular medium with an apparatus comprising a probe rod inserted into the solid medium and a processor, said probe rod carrying at least one photoemitter and at least one photodetector, forms the subject matter of the invention,
wherein the method comprises:
by the photoemitter, emitting a probe light signal towards the solid medium, said probe light signal producing, upon interaction with the solid medium, a return light signal scattered by the solid medium and having intensity fluctuations deriving from local inhomogeneities or anisotropies of the solid medium,
by the photodetector, measuring a plurality of intensity values of the return light signal in a plurality of respective detection points, in such a way as to detect said intensity fluctuations,
by the photodetector, measuring a plurality of intensity values of the return light signal in a plurality of respective detection points, in such a way as to detect said intensity fluctuations.

Also forming a subject matter of the invention is an apparatus for the optical characterization of a non-+homogeneous solid granular medium, said apparatus comprising:
a probe rod configured to be inserted into the solid medium, said probe rod carrying at least one photoemitter and at least one photodetector, wherein the photoemitter is configured to emit a probe light signal toward the solid medium, said probe light signal producing, upon interaction with the solid medium, a return light signal scattered by the solid medium and having intensity fluctuations deriving from local inhomogeneities or anisotropies of the solid medium, wherein the photodetector is operable to measure a plurality of intensity values of the return light signal in a plurality of respective detection points, in such a way as to detect said intensity fluctuations, and
a processor programmed to determine physical properties of the solid medium correlated to said intensity fluctuations, by statistical analysis of the plurality of intensity values of the return light signal.

The present invention exploits the phenomenon that occurs when a light radiation propagates in a non-homogeneous medium, within which it undergoes continuous effects of refraction, reflection, diffraction and scattering, globally called in the following interactions, which cause disuniformities and anisotropies of the intensity of said radiation in the medium. These inhomogeneities and anisotropies possess spatial characteristics that depend on the structures present in the snowpack and the related inhomogeneities dependent on grain shapes and structures. By characterizing these inhomogeneities and anisotropies and their spatial characteristics, properties of the medium may be measured that provide statistical information about the size and distribution of the inhomogeneities.

The properties that may be characterized according to the invention may relate to the size, size distribution, shape, structure, and arrangement of the elements that make up the inhomogeneities of the medium in which light radiation propagates and may be used to derive information about the mechanical properties of the medium based on the properties of the objects of snow and/or ice that make up the medium. In particular, these properties may be related to the stability of the medium for an assessment of the stresses that may change it and/or make it unstable.

### DETAILED DESCRIPTION OF THE INVENTION

Further features and advantages of the invention will become clearer from the following detailed description of an embodiment of the invention, made with reference to the accompanying drawings, provided purely for illustrative and non-limiting purposes, in which

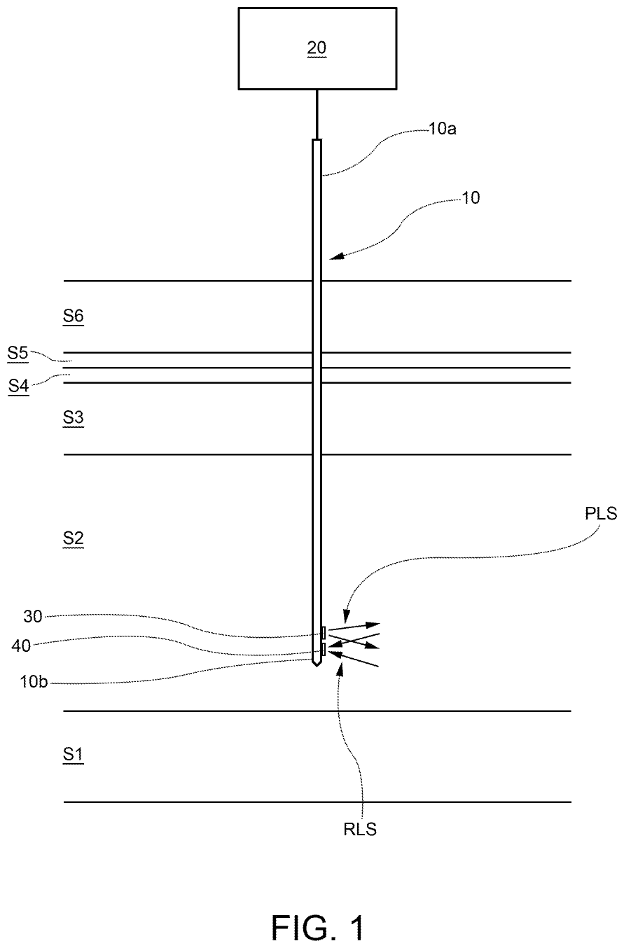

FIG. 1 is a schematic representation of a snowpack in which a probe rod of an apparatus according to the invention is inserted,

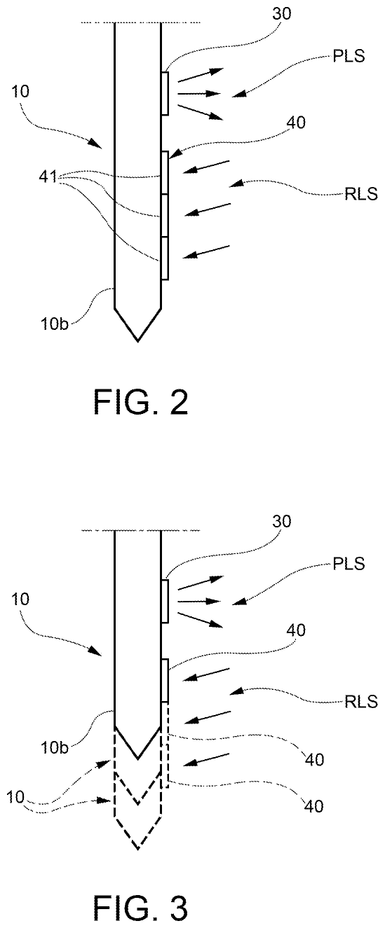

FIGS. 2, 3 and 4 are schematic representations of the probe rod in different embodiments,

FIGS. 5a and 5b are views from different sides of one end of the probe rod according to an embodiment,

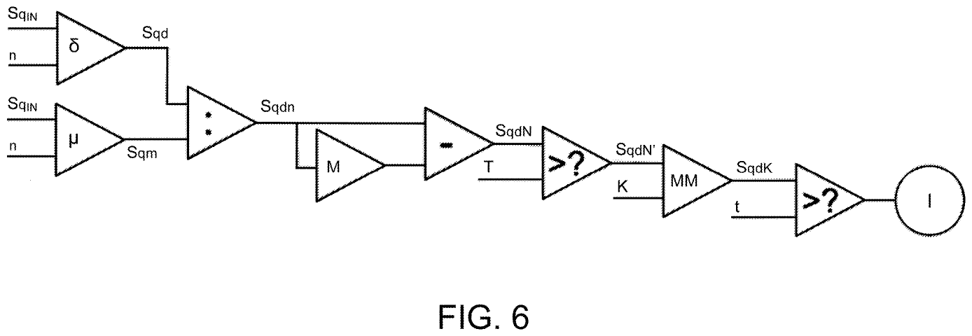

FIG. 6 is a flow diagram of an applicable calculation method according to the invention.

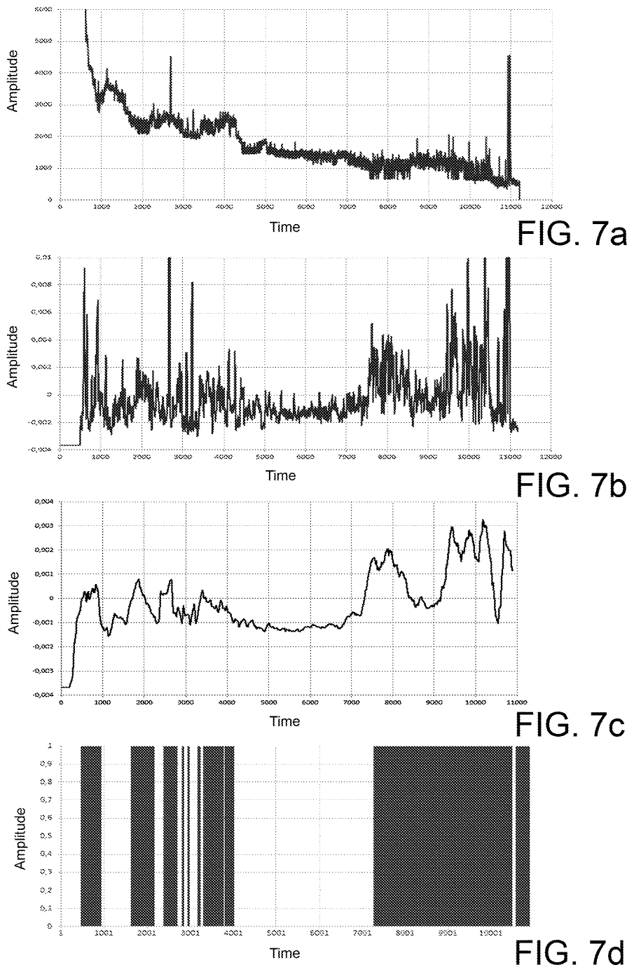

FIG. 7a through 7d are graphs showing an example of a sequence of measured intensity values (FIG. 7a) and further sequences of values (FIG. 7b-7d) obtainable, from the initial sequence, after respective calculation operations of the method in FIG. 6. In the graphs, the ordinate shows the amplitude in arbitrary units, and the abscissa shows the time in arbitrary units, and thus the depth in the medium (assuming the probe rod is advanced at constant speed),

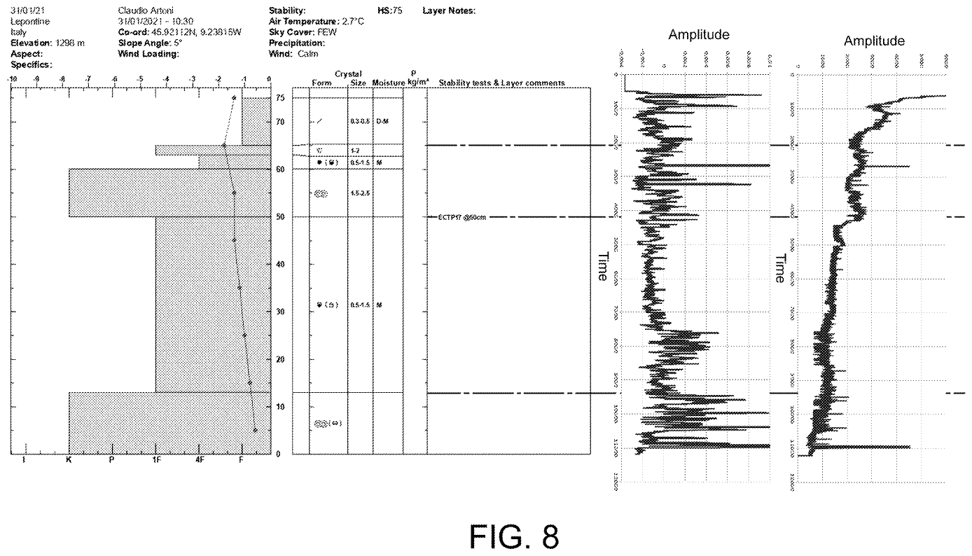

FIG. 8 depicts, in the left panel, a stratigraphic profile observed manually and visualized with the Snow Pilot software, and, in the left panels, a first graph showing the intensity of the measured light signal as a function of time (corresponding to the graph in FIG. 7a), and a second graph showing the variance of the same signal, normalized to the intensity (corresponding to the graph in FIG. 7b).

FIG. 1 shows a probe rod 10 inserted into a solid granular medium, such as a snowpack, comprising one or more layers arranged at different depths, and denoted in the figure with references S1 to S6. 10a and 10b denote respectively the opposite ends of the probe rod 10. A first end 10a remains, in use, generally outside the solid medium to be investigated, to allow manipulation of the probe rod 10; this first end 10a will be referred to hereinafter as the control end. The second end 10b is in use immersed in the solid medium to be investigated; this second end 10b will be referred to hereinafter as the head end. The probe rod 10 is connected by wire or wirelessly to a processor 20. The probe rod 10 carries at least one photoemitter 30 and at least one photodetector 40. The photoemitter 30 is configured to emit a probe light signal PLS to the solid medium S1-S6. Such probe light signal produces, upon interaction with the solid medium S1-S6, a diffused return light signal RLS from the solid medium S1-S6 that has fluctuations in intensity resulting from local inhomogeneities or anisotropies of the solid medium S1-S6. The photoemitter 30 may comprise one or more lasers, capable of emitting radiation in the visible or infrared range. The photodetector 40 is operable to measure a plurality of intensity values of the return light signal at a plurality of respective detection points, so as to detect the aforesaid intensity fluctuations.

The propagation of radiation in the snowpack occurs as commonly described for the propagation of radiation in a medium commonly referred to as “optically thick,” that is, a medium in which the distance corresponding to the thickness of the medium and/or the distance along which the radiation propagates greatly exceeds the mean distance between two successive interactions, also called the mean free path. This results in a substantial loss of directionality of the radiation in the medium, which propagates in a manner substantially diffusive in all directions and with substantially uniform intensity when averaged over spatial scales much larger than the size of the structures, but which tends to decrease as it moves away from the source.

A sensor medium that measures the radiation intensity on spatial scales on the order of or smaller than the size of the intensity fluctuations in the medium provides the information needed to characterize the properties of the snowpack based on the properties of its constituent grains. Measurements made at different locations allow information on the stratification of the snowpack to be traced, similar to that which is obtained by the conventional stratigraphic analysis described above.

In general, two methods may be envisioned to approach the study of grains, based on the characterization of disuniformities and the characterization of radiation anisotropies in the medium, respectively, as detailed hereinafter. Any embodiment that exploits one or both of these methods is part of the present invention.

In particular, two approaches may be envisioned for characterizing intensity fluctuations, whether derived from intensity inhomogeneities, or derived from the anisotropies thereof, or both. A first approach, exemplified in FIG. 3, is to use sensor means that move within the snowpack. In the example in FIG. 3, the probe rod 10 moves, taking the photodetector 40 therewith (the various positions of the tip of the probe rod 10 and the photodetector 40 are shown with a dotted line). The photodetector 40 then moves to successive detection points, thereby measuring intensity values that vary over time depending on the position of said sensor at the instant of measurement. These variations may be appropriately related to the spatial fluctuations in intensity or the anisotropies with which the radiation propagates in the snowpack, as described in detail hereinafter.

A second approach, exemplified in FIG. 2, is to use sensor means that are able to measure a multiplicity of intensity values corresponding to radiation intensity values originating from different locations in the snowpack, or having different directions of origin. In the example in FIG. 2, the photodetector 40 is configured as an array of sensing elements 41 such as a CCD or CMOS sensor, and therefore intrinsically comprises a plurality of detection points.

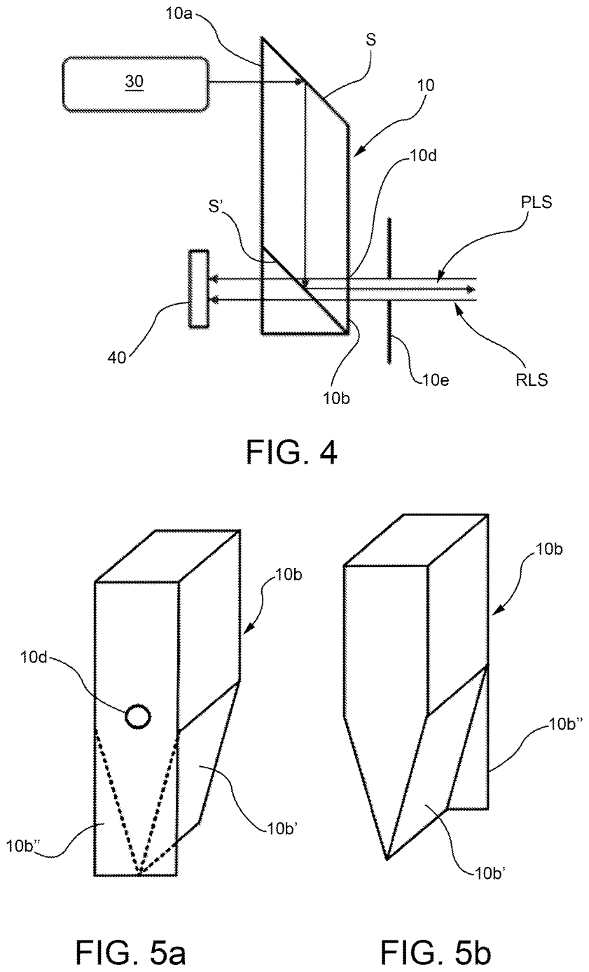

FIG. 4 shows a further embodiment of the probe rod 10. In such an embodiment, the photoemitter 30 is placed at the control end 10a of the probe rod 10, or at an intermediate point between the control end 10a and the head end 10b. An optical system 10c, provided for guiding the probe light signal PLS emitted by the photoemitter 30 to an optical window 10d arranged on the probe rod 10 and through which the probe light signal PLS is radiated to the solid medium, is arranged on the probe rod 10. The same optical window 10d allows for collecting the return light signal RLS, which finally reaches the photodetector 40. In the example shown, such an optical system is exemplified by a first surface S, arranged at the photoemitter 30 and that reflects the light emitted by the photoemitter 30 back to the head end 10b of the probe rod 10, and a second surface S′, arranged at the optical window 10d and that further reflects the light back to the optical window 10d. Such second surface S′ is then penetrated by the return light signal RLS that has passed through the optical window 10d to reach the photodetector 40. The surfaces S and S′ may be implemented on a light guide, such as an optical fiber. A diaphragm 10e may be associated with the optical window 10d to limit the angular width of the light collected by the photodetector 40 through the optical window 10d. In FIG. 4, the photoemitter 30, the photodetector 40, and the diaphragm 10e are for simplicity depicted detached from the probe rod 10, although in reality they are all on board said probe rod. It is further understood that elements discussed separately in the various embodiments in FIG. 2-4 may be combined with each other.

FIGS. 5a and 5b show an embodiment equipped with a particular profile of the head end 10b of the probe rod 10. Such profile comprises a wedge portion 10b′ that ensures the penetration of the probe rod 10 by imposing a lateral displacement of the medium, and a cutting plane or blade 10b″ arranged alongside the wedge portion 10b′, which preserves the section along which the lighting and light-receiving window 10d runs (the window 10d is arranged flush with one face of the cutting blade 10b′). The edge of the cutting blade 10b′ is placed orthogonally to the apical edge of the wedge portion 10b′. As becomes clear to a person skilled in the art, the smaller the size of the measurement head, the less disturbance imposed on the snowpack.

The procedures for analyzing intensity values depend on the manner in which the radiation is sent to and collected by the device and may be distinguished into two classes: 1) measurement of the spatial distribution of intensity fluctuations, including by measuring fluctuations in time recorded at different positions of the sensor means; 2) measurement of the propagation directions of the radiation from the medium under study.

Light radiation may be sent into the medium from a fixed or variable position, inside or outside said medium. In particular, the radiation may be sent from a position that is kept substantially fixed with respect to the sensor means. The radiation may be sent into the medium according to one or more predetermined directions and may be collected by the sensor means according to one or more predetermined directions. The radiation emitted by the source may be measured in intensity by means of a device that provides its received power at each instant in which a measurement is collected, so that the values of the collected signals may be properly related to the actual value of the power emitted by the source.

The sensor means may usefully be arranged to be sensitive to different regions of the spectrum, overlapping each other or not.

The present invention includes embodiments in which the sensor means are suitable for determining intensity values of the radiation coming from one or more positions in the medium under study, as well as intensity values of the radiation coming from one or more directions of origin, possibly by the use of suitable optical devices.

A possible embodiment of the present invention is obtained by way of example by means of a device arranged to be moved within the medium under study measuring radiation intensity values that depend on the position of the device at the instant of time when the measurement is made. The device contains a light source 30 preferably a laser, which illuminates the medium, and a sensor device 40 with an element sensitive to the radiation emitted by the light source, which measures the intensity of the radiation from a limited range of source directions. The light is sent into the medium and received by the medium through optical windows. Said optical windows may be coincident. The entire device is configured in such a way as to change the structure of the medium under study as little as possible during movements. The propagation of the radiation is affected by the inhomogeneities of the medium and is influenced thereby depending on the characteristics of size, shape and composition, changing the intensity values measured by the sensor means depending on the position of the device at a given instant of time when the measurement is made. By moving the device, the sensor means are then illuminated with radiation that fluctuates in intensity depending on the disuniformities or anisotropies of the radiation imposed by the inhomogeneities of the medium. The time series analysis of the measurements of the intensity values and the fluctuations thereof provides information on spatial inhomogeneities of the intensity of the radiation in the medium. The analysis of said inhomogeneities provides information on the inhomogeneities of the medium.

Another embodiment of the present invention is obtained, for example, by a device arranged to operate within the medium under study, said device having sensor means that measure a multiplicity of radiation intensity values from a multiplicity of positions within the medium. Such correspondence is preferably accomplished by means of an optical system. A variant of this embodiment is achieved by an optical system that associates each sensor means with a direction of propagation of the radiation from the medium under study.

A further variant is obtained by means of an optical system that forms a combination of the preceding two variants. Fluctuations in intensity relative to different positions or directions of propagation depend on the embodiment, which remains fixed, and the disuniformities or anisotropies of the radiation imposed by the inhomogeneities of the medium. The distributions of intensity and the fluctuations thereof measured by the device may be considered as suitable images produced from the disuniformities or anisotropies of the radiation. The analysis of said images provides information on the inhomogeneities in the medium.

The sensor means are connected to an acquisition system (the processor 20) that converts the signals from the sensor means into a data set of measured intensity values.

The data measured through the sensor means are used to derive the mean intensity and the fluctuations thereof, both referring to appropriate spatial intervals in the medium. Various statistical properties used to quantify these two characteristics of the intensity of the radiation in the medium, as well as different combinations thereof, may provide useful information for the determination of the properties according to the present invention. Purely by way of example, it is reported that the intensity fluctuations generally show a spatial structure related to the spatial structure of the inhomogeneities in the medium under study, which therefore may be determined by both embodiments discussed above. Moreover, the mean intensity values depend on the ways in which the radiation propagates in the medium within which it is subjected to continuous events of refraction, reflection, diffusion, and absorption by the constituent elements of the medium under study.

The determination of the properties of the medium and the constituent objects may be carried out by computational means based on procedures of statistical data analysis relating to appropriate spatial intervals in the medium, including, purely by way of example, means, variances, or on quantities derived from Fourier transforms and/or correlation functions. The data may be normalized or compensated if necessary according to appropriate statistical analysis procedures.

Purely by way of example, an example of an algorithm that may be used according to the present invention is given, with reference to FIG. 6, which represents a flowchart of the operations that the processor 20 may perform. The values of the parameters that were used in the implementation of the algorithm that led to the results shown in the example discussed hereinafter are given in parentheses.

A sequence Sq_{IN} of N (11000) measured intensity values, an example of which is shown in FIG. 7a, is processed according to the following operations. Such measured intensity values were taken at respective points at different depths within the medium. As described above, this may be achieved by moving the probe rod 10 vertically within the medium and taking a measurement of the light intensity received by the photodetector 40 at the respective position reached.

The standard deviations σ over N−n+1 intervals of length n (20) defined as input are calculated. This yields a sequence Sqd of N−n+1 values (10981) of standard deviation. The averages μ of the sequence of values Sq_{IN} over the N−n+1 interval of length n are also calculated, yielding a sequence Sqm of N−n+1 mean values (10981). The values of the sequence Sqd are divided by the values of the sequence Sqm, resulting in a sequence Sqdn of N−n+1 standard deviation values normalized to the local mean (shown in FIG. 7b). The mean M of all the values in the normalized standard deviation sequence Sqdn is calculated, and this mean M is subtracted from the normalized standard deviation sequence Sqdn, yielding a sequence SqdN of N−n+1 normalized standard deviation values, from which the mean M has been subtracted. If necessary, for example, because of the presence of very pronounced peak values in the sequence, the sequence values SqdN are compared to a threshold value T (0.01): all values greater than the threshold are replaced with the value T of said threshold, resulting in a sequence SqdN′. On the values of the sequence SdqN, or SqdN′, a movable mean MM is performed on N−n−k+2 intervals of length k (300), corresponding to a layer of thickness D (in the example discussed below, where the depth of the examined medium is about 0.9 m, the thickness D is about 20 mm), thus obtaining a sequence Sqdk of N−n−k+2 values (depicted in FIG. 7c). The numerical values of the sequence Sqdk are positive and negative and provide information, respectively, on the greater or lesser presence of intensity fluctuations within each interval with spatial resolution D. By comparing the sequence values Sqdk with a threshold t (0), a discriminant is obtained that provides information I on the change in geometric properties of the grains that resulted in the measured fluctuations, consequently on the change in physical properties of the snowpack between layers (depicted in FIG. 7d).

By virtue of the high temporal acquisition resolution, it is possible to vary the analysis parameters, e.g., the lengths n and k of the analysis intervals may be chosen to be different from the values indicated above. In this way, for example, grain size and spatial resolution may be chosen, the scale sizes of which correspond to the product between the probe speed and the time corresponding to the length n and the product between the probe speed and the time corresponding to length k, respectively. One may further improve the analysis by repeating it for different values of n, in such a way as to systematically select the scale lengths to which the system is sensitive from time to time.

Based on the values obtained in the sequence Sqdk and operating with more than one threshold t, it is also possible to obtain information on different scale lengths present in the medium.

Note that the sequence Sqdk may be further exploited for the evaluation of particle size variation.

The algorithm described here is purely by way of example. As will appear evident to a person skilled in the art, any statistical analysis method based on, for example, correlation functions, Fourier analysis, frequency filters, and template matching may advantageously be exploited to implement the present invention in the recognition of morphological properties that determine the sequence Sq_{IN} of measured intensity values.

The comparison in FIG. 8 of the result of the analysis performed with the algorithm described above shows a region (approximately between the abscissa values 7200 and 9000, corresponding to 25-15 cm from the base) that apparently does not match the information obtained from the snowpit. This may be explained by a different way of analyzing the data.

In fact, the use of statistical analysis, whether Fourier or correlation or otherwise, also allows grain size information to be derived in many cases. The length over which the light intensity remains substantially constant is statistically indicative of the size of the snow and ice grains that populate the material near the measurement window. Information on this length may be obtained statistically by means of the correlation function, the characteristic time of which, converted to length by means of the probe velocity, provides information on grain size, as is well known to persons skilled in the art. In the example given here, the characteristic width of the correlation functions has been quantified by the standard deviation of the function normalized to the maximum thereof, and this compared with the grain size measured on the crystallographic tablet by a calibration procedure based on direct measurements of the grains of the snowpit carried out simultaneously with the measurements conducted with the probe. A proportionality constant C₀ is derived between the width of the correlation function and the actual grain size.

The algorithm used for grain size evaluation in the case in example is as follows. A subsequence sq_{IN} of m (500) values is extracted from the sequence Sq_{IN} of N (11000) measured intensity values. The extension of the subsequence sq_{IN} must be large enough to contain a representative sample of fluctuations to carry out the analysis (typically tens or hundreds of fluctuations), but at the same time not too large so as not to include regions of the probed layer that exhibit different physical characteristics. The Fourier transform of the subsequence sq_{IN} is carried out, for example by an algorithm known as fast Fourier transform FFT or the extensions thereof. The zero-frequency contribution is removed, resulting in a sequence of values sq₁ the squared modulus of which is calculated, thus obtaining the power spectrum PSD. An additional Fourier transform (or inverse transform) of the spectrum PSD is performed, obtaining a sequence sq₂ that provides the autocorrelation function of the subsequence of values sq_{IN}. The sequence sq₂ is normalized by dividing it by the maximum value obtained at zero movement, yielding the normalized sequence sqn. The width of the correlation function is then calculated by averaging the quadratic deviations weighted with the values of said function, obtaining the width value d. Such value is converted to distance on the basis of the vertical distance between two successive values measured by the probe, derived on the basis of the acquisition frequency and velocity of the probe in the medium (150 microns in the case under consideration). The result is multiplied by the conversion constant C₀ corresponding to the calibration performed by direct measurement in a known snowpit.

By way of example, the results obtained by analyzing the correlation functions evaluated on the data in the example in FIG. 8 for different values of depth are shown.

TABLE 1

| Depth | 70 | 65 | 60 | 50 | 40 | 30 | 20 | 10 |
|---|---|---|---|---|---|---|---|---|
| Size | 0.4 | 0.4 | 1.2 | 1.2 | 0.5 | 0.6 | 0.6 | 0.9 |

The values reported express the average grain size in mm, and the depth is reported in meters from the base, as is customary in the snowpit. The agreement with values obtained from direct grain measurements, where available, demonstrates the effectiveness of the method. It also explains the discrepancy highlighted above, which shows that the grain size is homogeneous up to 10-15 cm above the ground, while the amplitude of fluctuations is increased as obtained in the preceding result. This shows that the information obtained with the previous algorithm is complementary to the grain information obtained above, providing information on how the grains themselves are distributed in space even at the same size. This information is of particular relevance to the evaluation of the mechanical properties of the medium, as is clear to persons skilled in the art.

### EXAMPLE

An apparatus was made with the geometry of FIGS. 5a and 5b, adapted to alter as little as possible the structure of the snow through which it is run. The measuring head is moved vertically, penetrating through the layers of the snowpack. It contains a laser, wavelength 640 nm, which horizontally illuminates the snow through an optical window that also serves to collect scattered light falling on a sensor, according to the embodiment shown in FIG. 4. The device is powered by a battery contained in an external portable unit, which contains a microcontroller programmed to acquire the light power data read by the sensor and also carries out data collection and recording on an SD card.

FIG. 8 shows an example of the data sets collected in the field in January and February 2021.

FIG. 8 further shows two graphs, both as a function of time, representing the coordinate that provides the depth at which the intensity value reaching the sensor is detected: 1) values of the instantaneous power reaching the sensor, from which the average value is subtracted; 2) values of the variance of the power values, calculated over an appropriate range of recorded values, normalized to the average power value measured over the same range. The spatial resolution of the data set was on the order of 0.12-0.15 mm (corresponding to 83.3-66.7 points per centimeter), depending on the regularity with which the penetration of the snowpack has been performed. The thickness thereof was about 0.9 m measured vertically.

In particular, the second graph provides information that was found to be correlated with ice grain size, obtained from a specific grain size measurement carried out according to the international prerequisites of the Avalanche Service, within a trench dug at the measurement carried out with the probe.

As may be seen from the figure, illustrative of numerous tests performed under different conditions, the detected values of the collected power vary as depth increases, decreasing due to the decrease in the solar component, which in the specific case was not negligible compared to the power of the laser used. It should be noted also that the variation does not follow a regular curve, demonstrating the presence of layers within which radiative transfer occurs differently. Furthermore, the normalized variance value also changes by more than an order of magnitude between strata, highlighting the diversity in the fine structure of the grains, which in some cases also appears to be correlated with the variation in collected power.

### Snowpit Analysis

The analysis carried out according to the protocol provided for avalanche prediction (snowpit) shows evidence of the presence of a basal layer up to 13 cm from the base composed of melt-freeze crust and ice grains (Ice Form). Provided above is a layer extending up to 50 cm composed of partially rounded, angular grains (FC-RG) with a typical size between 0.5 and 1.5 mm. This layer is overlaid with a melt-freeze crust between 50 cm and 60 cm, with larger grains, between 1.5 and 2.5 cm, packed with consolidation necks. The mechanical detachment test (Extended Column Test) showed a planar fracture below the crust after 17 hits (ECTP17). Above them rest two other thinner homogeneous layers composed of rounded grains ranging in size from 0.5 to 2 mm similar to the 13 to 50 cm layer. A layer of partially decomposed fresh snow concludes the profile. The histograms highlight the response to a standard test called the “hand test” for assessing layer hardness. Thus observed is the division of the snowpack into a surface layer of partially decomposed, soft fresh snow (hardness F); an intermediate layer of rounded grains with a medium hardness (1F); and two crusts of high hardness (K) below 13 cm and between 50 and 60 cm. The relevant layer for detachment is the one at 50 cm.

Comparison with the Data Collected by the Probe

The graph for the data collected at the snowpit shown in FIG. 8 shows the normalized variance values along the stratigraphic profile. It should be noted that since there is no absolute reference for the vertical position, the vertical coordinates are for now only a time reference automatically acquired by the probe during the descent through the snowpack. The rate of descent was kept constant enough that time values could be interpreted as proportional to depth values.

The normalized variance analysis in the graph above shows the division in the three main strata also obtained in the snowpit. The sensitivity to the presence of the two freeze crusts and the difference with the intermediate layer, especially the transition to the layer placed at 50 cm at which the detachment test ECT was successful, is evident.

The normalized variance value also changes more than an order of magnitude between strata, as also evidenced by the comparison with the snowpit shown in FIG. 8. This highlights the diversity in the fine structure of the grains, which in some cases is also correlated with the variation in the power collected, and thus determines the observable used by the probe described in WO 2017/058929 A1. It should be noted also that the latter identifies layers based on the intensity value, so it only highlights layer surfaces. The variance method, on the other hand, obtains information on the intensity statistics within said layer, thus allowing the properties of the grains to be traced at all depths, even when the intensity value is constant or no significant variation is appreciated. The method thus obtains information about the strata from the changes in the fluctuations as the depth changes, while it obtains information about the grains (not about their size) from the magnitude of said fluctuations.

Finally, the measurement of the properties of the objects constituting the non-homogeneous medium is used to derive quantities related to mechanical properties of the medium, such as compressive strength, tensile strength, displacement strength, or resistance to temperature changes that may change the internal structure of the medium. In particular, the properties of said objects may vary with the position in the medium and even more particularly may vary with depth, even in distinct layers. The characterization of properties depending on the position, particularly along the essentially vertical direction, provides additional information about the mechanical properties of the medium, as is well known to persons skilled in the art.

What is claimed is:

1. A method for the optical characterization of a non-homogeneous solid granular medium, with an apparatus comprising a probe rod inserted into the non-homogeneous solid granular medium and a processor, the probe rod carrying at least one photoemitter and at least one photodetector,
the method comprising:
a) by the photoemitter, emitting a probe light signal towards the non-homogeneous solid granular medium, the probe light signal producing, upon interaction with the non-homogeneous solid granular medium, a return light signal scattered by the non-homogeneous solid granular medium and having intensity fluctuations deriving from local inhomogeneities or anisotropies of the non-homogeneous solid granular medium,
b) by the photodetector, measuring a plurality of intensity values of the return light signal in a plurality of respective detection points, in such a way as to detect the intensity fluctuations, and
c) by the processor, determining physical properties of the non-homogeneous solid granular medium correlated to the intensity fluctuations, by statistical analysis of the plurality of measured intensity values of the return light signal.

2. The method of claim 1, wherein the statistical analysis comprises Fourier and correlation analyses.

3. The method of claim 2, wherein step c) comprises
extracting a subsequence from the plurality of measured intensity values of the return light signal, the subsequence containing a representative sample of the intensity fluctuations,
performing a Fourier transform of the subsequence,
calculating a PSD power spectrum from the Fourier transform of the subsequence, and
calculating, by the Fourier transform, an autocorrelation function of the subsequence.

4. The method of claim 3, further comprising calculating a width of the autocorrelation function.

5. The method of claim 1, wherein step c) comprises
calculating a plurality of local variance values and a plurality of local mean values from the plurality of measured intensity values, and
calculating a plurality of normalized variance values by dividing the local variance values by the local mean values.

6. The method of claim 1, wherein the photodetector is configured as an array of sensing elements, each defining a respective detection point of the plurality of detection points.

7. The method of claim 1, wherein the probe rod is moved in the non-homogeneous solid granular medium, and the detection points are defined by respective positions of the photodetector.

8. The method of claim 1, wherein the probe rod comprises at least one optical window through which the probe light signal is transmitted towards the non-homogeneous solid granular medium, and through which the return light signal is transmitted towards the photodetector.

9. The method of claim 1, wherein the probe rod comprises a head end comprising a wedge portion and a cutting blade arranged alongside of the wedge portion.

10. The method of claim 1, wherein the non-homogeneous solid granular medium is a snowpack.

11. The method of claim 1, wherein step c) comprises identifying interfaces between layers of the non-homogeneous solid granular medium having different physical properties.

12. The method of claim 1, wherein step c) comprises estimating an approximated size of grains of the non-homogeneous solid granular medium.

13. The method of claim 12, wherein the estimating comprises performing a calibration by direct measurement of the size of the grains of the non-homogeneous solid granular medium.

14. An apparatus for the optical characterization of a non-homogeneous solid granular medium, the apparatus comprising:
a probe rod configured for insertion into the non-homogeneous solid granular medium, the probe rod carrying at least one photoemitter and at least one photodetector, wherein the photoemitter is configured to emit a probe light signal towards the non-homogeneous solid granular medium, the probe light signal producing, upon interaction with the non-homogeneous solid granular medium, a return light signal scattered by the non-homogeneous solid granular medium and having intensity fluctuations deriving from local inhomogeneities or anisotropies of the non-homogeneous solid granular medium, wherein the photodetector is operable to measure a plurality of intensity values of the return light signal in a plurality of respective detection points, in such a way as to detect the intensity fluctuations, and
a processor programmed for determining physical properties of the non-homogeneous solid granular medium correlated to the intensity fluctuations by statistical analysis of the plurality of measured intensity values of the return light signal.
